(19) United States
(12) Patent Application Publication
(10) Pub. No.: US 2011/0200165 A1
(43) Pub. Date: Aug. 18, 2011

(54) CONTRAST AGENT-REINFORCED RADIOTHERAPY HAVING HIGH-OUTPUT TUBES

(75) Inventor: Hubertus Pietsch, Kleinmachnow (DE)

(73) Assignee: BAYER SCHERING PHARMA AKTIENGESELLSCHAFT, Berlin (DE)

(21) Appl. No.: 13/054,270

(22) PCT Filed: Jul. 7, 2009
(86) PCT No.: PCT/EP2009/004893

§ 371 Date: Apr. 15, 2011

(30) Foreign Application Priority Data
Jul. 16, 2008 (DE) 10 2008 034 020.0

(51) Int. Cl. A61N 5/10; A61B 6/03

(52) U.S. Cl. 378/14; 378/65; 378/4

(57) ABSTRACT

The invention relates to the combination of intravascularly administered contrast media and low-energy x-ray radiation for radiation-therapeutic treatment of tumors. The contrast medium substances contain at least one radiation-absorbing element and are used for diagnosis and for a photoelectrically activatable dose increase in therapy.

The invention relates to the combination of intravascularly administered contrast media and low-energy x-ray radiation for radiation-therapeutic treatment of tumors. The contrast medium substances contain at least one radiation-absorbing element and are used for diagnosis and for photoelectrically activatable dose increase in therapy.

### PRIOR ART

Radiation therapy is one of the pillars of the treatment of ontological diseases. A successful radiation-therapeutical treatment of tumors requires early diagnosis and localization thereof. The purpose is to focus a high radiation dose, sufficient to kill the tumor, on the tumor and thus to kill all tumor cells without damaging the surrounding healthy tissue.

For radiation therapy, linear accelerators with high energy up to 20 MeV are now used. The radiation in the form of photons or electrons is concentrated by static or dynamic diaphragm systems (collimators) on the tumor area, so that the surrounding healthy tissue is preserved. An exception is the total-brain irradiation, which is used in the case of multiple brain metastases.

To optimize the dose distribution, multi-field techniques are used, in which the target volume is placed in the area of overlap of several radiation fields (conformal radiation therapy). A new process is the intensity-modulated radiation therapy in which in addition to the field limitation, the radiation dose is also modified within the field.

In radiation therapy, the treatment of the tumor is performed according to the irradiation planning. This requires precise adaptation of the patient's positioning to the irradiation planning before each therapy session. In addition to immobilization techniques, imaging techniques are also increasingly used in this respect. To this end, linear accelerators are equipped with additional imaging units, with whose assistance the correct positioning of the target volume can be verified (1).

Since tumor cells exhibit a reduced capacity for repair of radiation damages, the irradiation is divided into many individual doses of 2-3 Gy, and the total dose is thus distributed over several weeks (fractionated radiation therapy). In selected cases, primarily in the case of small brain tumors, the entire radiation dose is also administered as a single dose (radiosurgery).

In addition to the established therapy with linear accelerators, there are more expensive techniques, such as irradiation with neutrons, protons or heavy particles. In the majority of cases, these units are located in large-scale research centers and have not yet found the way to routine use. The irradiation from outside (teletherapy) is supported by interstitial administration forms in which radioactive implants are placed permanently or temporarily in the target volume (brachytherapy).

An important requirement for a successful radiation therapy is the irradiation planning. On the basis of CT images, a three-dimensional model of the tumor region is made, with whose assistance possible irradiation processes are optimized with computer support. For physical reasons, different x-ray energies are used for CT imaging and for radiation therapy. For CT images, the range of up to a maximum 140 keV is used, while the lower energies in the therapy begin only at 1 MeV. This has the result that specifically the modern irradiation units are not suitable for a high-resolution imaging. Conversely, x-ray units with acceleration voltages of up to 140 kV, which are extremely well suited for the imaging, have been replaced first by Telekobald and then by the high-voltage linear accelerator in conventional radiation therapy because of the shallow depth of penetration. Thus, the above-mentioned tomotherapy units, i.e., systems, which can be used in a like manner for imaging as for radiation therapy, are being considered at this time.

Computer tomography is a very widespread and highly precise radiological diagnosis technique. Very quick imaging with high local resolution is now possible via the enormous technological developments in recent years. The increase of both the speed of rotation (turn-around times 0.3-0.75 s) and the detector width (16-320 parallel lines) called for the development of new, extremely powerful x-ray tubes. These make possible the imaging of large anatomical areas (e.g., full-body CT) or functional parameters (e.g., perfusion) without cooling-off periods occurring.

The x-ray imaging is based on the different absorption properties of various types of tissue. These differences are especially pronounced between bones and soft tissue. For differentiation of soft parts as well as for visualization of organs, x-ray contrast media, which in most cases contain iodine as an absorbing element, are used. The latter locally increase the absorption of radiation. The x-ray contrast media approved for use in humans are extracellular substances with a small molecule size, which contain iodine as an absorbing element. As a result, the latter are distributed almost exclusively passively with the liquid stream and selectively move into those spaces that are connected to the site of application by open pores or other accesses (2). The excretion is carried out renally via passive glomerular filtration. Systematically administered intravenously or intraarterially, these substances also accumulate in tumors because of their pharmacokinetic properties. This characteristic is especially pronounced in brain tumors and brain metastases. The contrast medium molecules accumulate almost selectively in the tumor tissue therefore because of the defective blood-brain barrier.

In the energy range of the x-ray diagnosis (10-140 keV), interactions occur between radiation and matter because of the photoelectric effect, the Compton effect, and the elastic scattering. The absorption of x-ray contrast media is dominated by the photoelectric effect, which in turn increases with the atomic number Z³ (FIG. 1). By the element with high Z contained in the contrast media (in most cases, iodine with Z=53), the probability of a photoelectric interaction thus clearly increases. The presence of x-ray contrast media therefore also leads to an increase of the radiation of photoelectrons, x-ray fluorescence and. Auger electrons associated with the photo effect. This brings about a local enhancement of the radiation dose in the immediate surroundings of the contrast medium molecule. The latter increases linearly with the proportion by weight of iodine in the tissue being considered. In the diagnostic mode, this secondary emission plays a secondary role because of the low radiation doses.

The influence of photoelectric dose enhancement on the patient dose in the contrast-medium-enhanced x-ray diagnosis was discussed for the first time by Callisen (3). The possibility of the specific use of the dose enhancement in the radiation therapy was later examined by the work groups of R. Fairchild and A. Norman (4) (5). The latter demonstrated the effectiveness of the methods in a pre-clinical study for treatment of brain tumors on a rabbit tumor model (6). In this case, an iodine-containing contrast medium was administered intravenously at a very high dosage (3.5 g of iodine/kg of body weight (b.w.)). A mean iodine-based signal increase of 82 HU was measured on the basis of CT images. Then, a radiation dose of 5 Gy with a dose rate of 0.32 Gy/minute was incorporated. This therapy session was repeated three times, which resulted in an increase in the mean survival time by 50%. Later, the use of a modified CT device for therapy was proposed (7) by the same work group. The latter contains additional collimators with which the CT fan beam is converted into a bundle of rays (pencil beam), whereby the therapeutic target area is always found in the center of rotation (U.S. Pat. No. 5,008,907). The dose rate that can be achieved with this device in human application, which is limited because of the limited power of the x-ray tubes or the cooling rate to at most 9 Gy/hour, is problematical, however. No information on cooling times was given, the mean dose rate was 0.15 Gy/minute.

Based on the very positive therapy results in the animal model, an initial clinical study on the CT-based radiation therapy of brain metastases was performed (8). In this Phase I study, the reliable application of this therapy modality could be demonstrated on humans. In each therapy session, 150 ml of contrast media in two phases (50% bolus, 50% infusion) was administered intravenously, and 5 Gy was administered within 45 minutes (0.11 Gy/minute). An alternative process is the contrast medium-supported stereotactical synchrotron radiotherapy, in which a contrast medium is used in combination with monochromatic synchrotron radiation. With this technique, successful animal-trial studies were performed at the European Synchrotron Center in Grenoble. In this case, the contrast medium was infused intravenously over one hour; in addition, a short contrast medium bolus was administered every 15 minutes. Altogether, an extremely high iodine dose of 7.6 g/kg of body weight was administered. The irradiation with a dose of 10 Gy was carried out within 45 minutes, which corresponds to 0.22 Gy/minute (9).

In all animal-trial studies performed, the radiation dose of a therapy session was incorporated within 15 to 45 minutes, in an application on humans within 45 minutes. The corresponding mean tumor-dose rates were between 0.22-0.32 Gy/minute for the animal-trial studies and 0.11 Gy/minute on humans.

The focusing of the radiation dose on the tumor can be carried out by the superposition of several fields. This can be carried out by x-ray devices with spatially adjustable x-ray tubes that have the ability to apply the radiation from various partial angles (CT, angiography, C-arm). Based on the rotation of the radiation source, a computer tomography offers ideal requirements. An identical principle was carried out with the tomotherapy in the high-energy range and applies today as the most modern process for the IMRT (10). The distribution of the radiation dose on CT can be simulated with Monte-Carlo methods. In a work by Mesa, dose simulations at 140 kV and different concentrations of contrast media were performed as a function of human CT data sets (11). For tumor iodine-concentrations of 5 mg/ml, a dose distribution in the area of the tumor tissue that is comparable to the Gold Standard (10 MV linear accelerator) was determined. A study of a work group presented recently at the European Synchrotron Center in Grenoble leads to a similar result. For iodine concentrations above 5 mg/ml, the dose distributions with an 85 keV synchrotron radiation are comparable to a 6 MV therapy (12). For a contrast-medium-enhanced radiation therapy, therefore, the highest possible contrast medium concentrations in the tumor are an elementary requirement. The studies that are presented are based on the theoretical assumption of a static contrast medium concentration. Real contrast medium concentrations are always dynamic processes, however.

The contrast medium kinetics can be influenced by the form of administration. In clinical tumor diagnosis, intravascular injections are used. Since extracellular contrast media already pass from the blood into the interstitial space during the first capillary passage, the arterial phase is measured during the first vascular passage for visualization. In this phase, the visualization of the vascularization of the tumors can be shown. In the subsequent portal-venous phase, primarily hypovascular tumors are shown in the abdomen. In the interstitial phase, the contrast medium is concentrated in the entire tumor tissue, which can be used for differential diagnosis relative to cysts (13). In the contrast medium-enhanced radiation therapy, in contrast to diagnosis, the focus is not placed on the contrast-rich visualization of tumor-specific concentration patterns but rather the achievement of high contrast medium concentrations in the tumor. In the experimental studies on animal models, the contrast media were therefore administered intravenously at very high dosages of between 3.5 and 7.6 mg of iodine/kg of body weight (6, 9). These dosages are considerably above the maximum, clinically recommended dose in the x-ray diagnosis of 1.5 g of iodine/kg of body weight. Only very little is known on the contrast medium administration schemes used in experimental studies to date (e.g., contrast medium flow, mono-phase, bi-phase, NaCl flush); comparative studies for optimizing parameters were not presented previously. An alternative possibility for the form of administration is the intratumoral administration, in which the contrast medium is sprayed directly into the tumor or the tumor edge by means of a needle (US 2004/0006254 A1). Another invasive method is the convection-enhanced contrast medium administration (14). The high invasiveness and the difficult-to-predict distribution of the contrast media pose a significant obstacle, however, to reliable clinical application. An intratumoral administration is used in the contrast medium-supported radiosurgery, an alternative process. There, the radiation dose should be introduced within 30 minutes, which makes necessary a high contrast medium concentration over this period (US 2004/0006254 A1). An optimization strategy for prolonging the concentration in the tumor is the change in pharmacokinetic properties of contrast media. In this case, primarily the water solubility and the size of the compounds play a decisive role. The use of larger contrast medium molecules or particles results in an intratumoral administration in an extension of the contrast medium concentration (WO 00/25819).

None of these references contains information or proposals on the combination of the contrast medium concentration in the tumor after intravascular administration and the introduction of a clinically relevant radiation dose.

A patent for the hardware portion of the treatment method with use of the x-ray optical module was filed with the Patent Office under AZ. 10 2007 018 102.9 on Apr. 16, 2007.

### Short Description of the Invention

The invention relates to the combination of intravascularly administered contrast media and low-energy x-ray radiation for radiation-therapeutic treatment of tumors. The contrast media contain at least one ray-absorbing element and are used for diagnosis and for photoelectrically activatable dose increase in therapy. An intravenous (i.v.) or intraarterial (i.a.) administration of contrast media leads to a concentration of these substances in the tumor area. Independently of the type of administration (i.v./i.a.) and speed of administration (flow rate) as well as the dosage, this dynamic process shows a time-limited contrast medium concentration range iii the target area that is suitable for the contrast medium-enhanced therapy. In this time window, a local, therapeutically active synergistic effect of contrast media and x-ray radiation takes place. In this case, the time window is to be selected so that a higher contrast medium concentration than in the surrounding healthy tissue is present in the tumor (target area) over the entire irradiation period. The radiation also has to lie within the previously or synchronously determined time window in the energy range of photoelectric interaction, and therefore diagnostic x-ray tubes with acceleration voltages of up to 140 kV are suitable. In computer tomography, modern high-power x-ray tubes with high photon flux and high anode cooling power are used. For the first time, the latter are able to administer a therapeutic dose in this energy range and in the available time window in the target volume. This therapy modality can be made clinically useful by x-ray devices with high-power tubes, which make possible irradiation from various spatial partial angles (CT, angiography, C-arm). Only high-power tubes make it facultatively possible, also by means of x-ray-optical modules, to focus on the optimum energy range of the contrast-medium-enhanced dose increase.

### DESCRIPTION OF THE FIGURES

FIG. 1: Absorption properties for scattering, Compton effect and photo effect of an iodine-containing contrast medium solution (iodine proportion 10%) as a function of the photon energy (source: http://physics.nist.gov/PhysRefData/XrayMassCoef/cover.html).

FIG. 2: Spectral dose enhancement SDE(E) for an iodine-tissue mixture (iodine proportion 1%).

FIG. 3: Photon fluence of a CT tube at 80, 120, 140 kV of acceleration voltage.

FIG. 4: Theoretical and experimentally determined photoelectric dose enhancement as a function of the iodine concentration at 140 kV.

FIG. 5: Relative HU profile (CT measurement) and simulated dose-profile at 2, 5 and 10 mgl/ml at 140 kV in an iodine-doped gel phantom.

FIG. 6: Time plot of the iodine concentration in tumors on the VX2 tumor model in a contrast medium flow of 0.1 and 4 ml/s and intravenous administration.

FIG. 7: Time plot of the iodine concentration in tumor and skin on the GS9L tumor model with intravenous administration over 3 or 6 minutes (mean value+/−SEM).

FIG. 8: Time plot of the iodine concentration in tumor, brain and blood on the GS9L tumor model with intra-arterial administration (mean value+/−SEM).

FIG. 9: Iodine concentration in the tumor, carotid artery and scalp on the GS9L tumor model as a function of the iodine dosage (mean value+/−SEM).

FIG. 10: Head phantom (left), CT images with central (middle) or peripheral ionization chamber insert (right).

FIG. 11: Relative dose distribution in the head phantom in conventional CT at 140 kV.

FIG. 12: Diagrammatic visualization of a dual-source CT for radiation therapy (tube a) and simultaneous imaging (tube b).

FIG. 13: Critical load curve of the Straton Z high-power tube with product version 08 or higher (source: Siemens Medical Solutions. Manual Straton Z. 2005).

FIG. 14: Survival curve according to Kaplan Meyer for a contrast-medium-enhanced radiation therapy in comparison to therapy without contrast media and an untreated control group on the GS9L animal model.

FIG. 15: Example of the time plot of the iodine concentration in tumors on the GS9L animal model With intravenous administration of 2 g of iodine/kg of body weight.

FIG. 16: Dosage charts of the rat head in a conventional CT at 140 kV and an iodine concentration of tumors of 0, 4.2 and 6.2 mg/ml (above); axial dose profile (below).

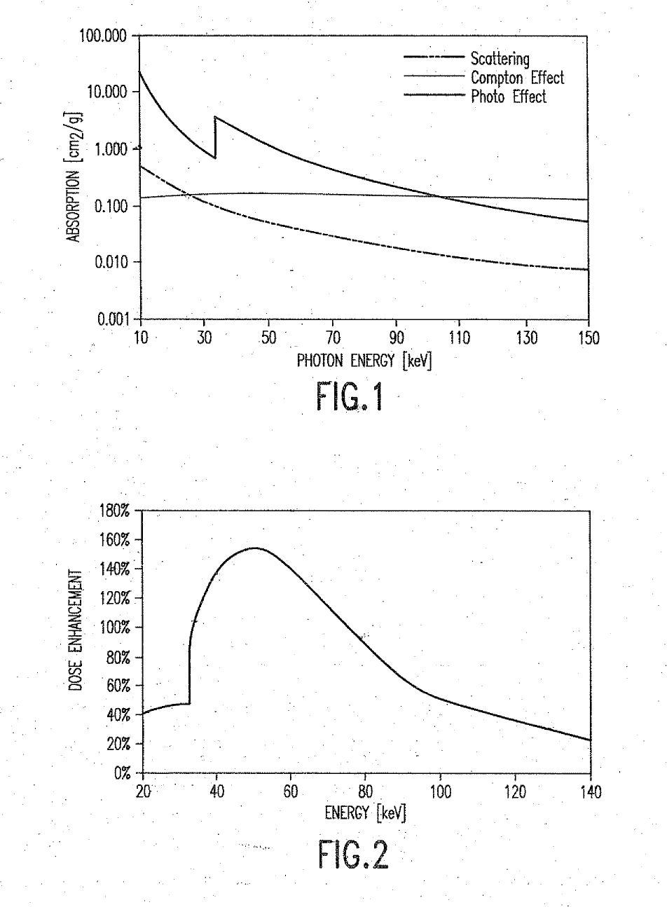

### DESCRIPTION OF THE INVENTION

### Photoelectric Dose Enhancement

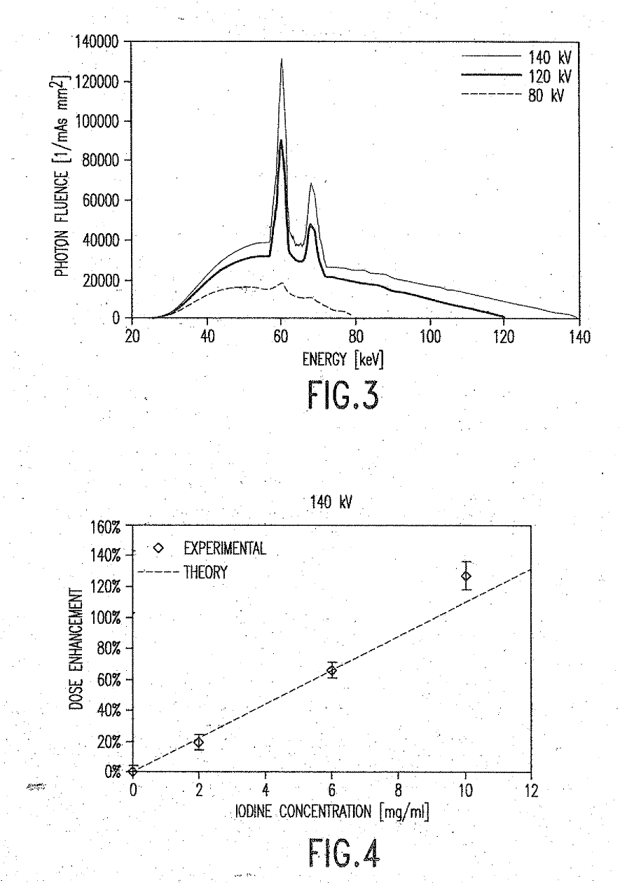

The tissue-absorbed radiation dose D is a function of photon fluence (φ| and the tissue-specific mass-energy transfer coefficients (μₑₙ/ρ):

$$D = {\int }_{E = 0 k eV}^{{E}_{max}} Φ ( E ) \frac{{μ}_{tr} ( E )}{ρ} d E , \qquad ( Equation 1 )$$

whereby E represents the energy of the photons in the tube spectrum (FIG. 3). For the energy range to 140 keV that is being considered, the energy losses caused by bremsstrahlung can be disregarded, so that μₑₙ/ρ can be replaced by the masses of energy-absorption coefficients (μₑₙ/μ)|.

Under the assumption of a constant φ|, the radiation dose is a function only of the material of specific coefficients μₑₙ(E)/ρ. The spectral dose enhancement SDE(E) can thus be described as the ratio of an iodine-tissue mixture relative to the pure tissue.

$$SDE ( E ) = \frac{{w ( \frac{{μ}_{en} ( E )}{ρ} )}^{Iodine} + ( 1 - w ) {( \frac{{μ}_{en} ( E )}{ρ} )}^{Tissue}}{{( \frac{{μ}_{en} ( E )}{ρ} )}^{Tissue}} , \qquad ( Equation 2 )$$

whereby w represents the fraction by weight of iodine. To calculate SDE(E), the μₑₙ(E)/ρ|coefficients of iodine and tissue (brain tissue ICRU-44) of the NIST reference database were used (15). A w of 1% was used, which corresponds to an iodine concentration of about 10 mg/ml. Up to this concentration, a start can be made from a linear connection between dose enhancement and iodine-mass concentration (in mg of iodine/ml). At higher concentrations, the differences in density between tissue and an iodine-tissue mixture can no longer be disregarded. The maximum dose enhancement is achieved at 50 keV; above 140 keV, the latter is hardly relevant any longer (FIG. 2). For an effective photoelectric dose enhancement, photon energies up to 140 keV are thus suitable. The latter correspond to the energy range of the x-ray diagnosis with tube voltages of between 8.0 and 140 kV (FIG. 3).

By the combination of equations 1 and 2, the dose enhancement DE can be quantified.

$$DE = {\int }_{E = 0 k eV}^{{E}_{max}} SDE ( E ) \cdot Φ ( E ) d E , \qquad ( Equation 3 )$$

When using a 140 kv tube spectrum, a dose enhancement of 109% (Table 1) is produced for an iodine-tissue mixture with iodine proportion of 1%.

TABLE 1
Dose Enhancement as a Function of Tube Voltage

| Voltage (kV) | 80 | 120 | 140 |
|---|---|---|---|
| DE/(1/(10 mgl/ml)) | 135% | 117% | 109% |

The energy of the radiation, i.e., the tube voltage, not only influences the photoelectric dose enhancement, but also the absorption of radiation in tissue. Since low-energy radiation proportions are clearly more greatly absorbed, a flatter depth dose plot is produced at 140 kV than at 80 kV. In the case of radiation therapy, a higher penetration depth or a reduction of the initial dose is thus produced.

For experimental verification of the dose enhancement, radiation-sensitive polymer gels were used (16). The latter were doped during the production with the dimeric x-ray contrast medium Isovist of different concentrations (corresponding to 0, 2, 6, and 10 mgl/ml). The irradiation of the gel dosimeter was carried out at a clinical CT (140 kV), and the analysis of the samples by means of MRT. The experimentally determined data produced a photoelectric dose enhancement of 12.2% per mg of iodine/ml (FIG. 4). In the area of measuring inaccuracies, this value is identical to the calculated value of 10.9% per mg of iodine/ml (FIG. 4).

For simulation of the spatial radiation dose distribution in the presence of iodine-containing contrast media, the Monte Carlo-based software ImpactMC (Vamp GmbH, Erlangen) was used. On the basis of CT images of a cylindrical gel phantom, the dose distribution was simulated with an iodine-doped core area. The size and absorption properties of the defined phantom materials are based on the ratios of a human head. In the cross-sectional dose profiles, a local increase of the radiation dose by 11.5% per mg of iodine/ml, limited to the iodine range, is shown (FIG. 5). In this case, a spiral-shaped irradiation of the entire phantom was simulated at 140 kV.

### Contrast Medium Concentration in the Tumor

In the case of intravascular administration, the concentration in tissues is determined by the perfusion, the permeability of vessels and the contrast medium excretion thereof. Tumors are well perfused in most cases because of the proliferating growth, and tumor vessels are characterized by a high permeability. This is true in particular for malignant tumors. In the brain, x-ray contrast media cannot leave the vessels because of the blood-brain barriers. Intracerebral lesions and tumors, however, exhibit a barrier disruption, by which contrast media are especially greatly and almost selectively accumulated there. In addition to the pharmacokinetic properties of the contrast medium and the physiological properties of the tissue, the contrast medium concentration can be influenced by the dosage and, within limits, also by the administration parameters.

In computer tomography, contrast media are in most cases administered intravenously via the arm veins. To this end, an injector is used via which the flow rate (ml of contrast medium or mg of iodine per s) and the period of the administration are preset. Both parameters together determine the contrast medium dose; 300 mg of iodine per kg of body weight (b.w.) is regarded as a standard dose. In clinical diagnosis, a total dose of 1.5 g of iodine per kg of b.w. should not be exceeded. Based on the viscosity of the substances as well as the vein stress, the flow rate is limited upward. Clinically, flow rates of between 1 and 8 ml/s are used.

Within these limitations, an optimization of the contrast medium administration is possible with respect to the diagnostic issue. In the contrast medium-enhanced radiation therapy, in contrast to the diagnosis, the contrast-rich visualization of tumor-specific concentration patterns is not emphasized, but rather the achievement of high contrast medium concentrations in the tumor in comparison to the surrounding healthy tissue. Under these conditions, the influence of the administration parameters (dose, flow rate) was still never examined extensively. In this case, flow rates of less than 1 ml/s with high contrast medium dosages (>1 g per kg of body weight) appear especially advantageous.

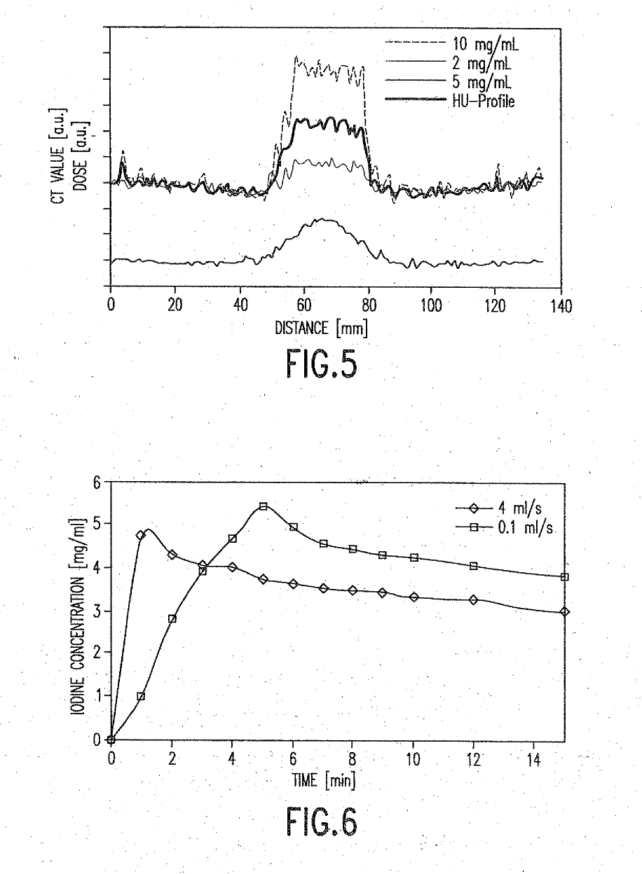

For typical examination of the influence of the contrast medium flow rate on the concentration in the tumor, a VX2 rabbit brain tumor model was used (17). A monomeric contrast medium (Ultravist 300, Bayer Schering Pharma, Berlin) was intravenously injected at a dosage of 2 g of iodine/kg of body weight. A quick administration (flow rate 4 ml/s) was compared to a slow contrast medium infusion (0.1 ml/s). To this end, the head of the tumor-bearing rabbit was examined at 6-hour intervals. In each case before and after the contrast medium administration, CT images were produced in the tumor area of the head. In the images, the mean increase of the absorption in the tumor (delta HU value relative to the Nativscan) and the tumor iodine-concentration based thereon Were determined at each point in time (0, 1, 2, . . . , 10, 12, 15, 20 minutes p.i.). A clear iodine concentration maximum in the tumor was noted both at high flow and at low flow (FIG. 6). At a flow of 4 ml/s, this was observed at one minute; at a flow of 0.1 ml/s, it was observed almost two minutes after the end of the contrast medium administration. The maximum concentration was 5.4 mg of I/ml with slower infusion, and 4.7 mg of I/ml at a higher flow rate. The time behavior of the drop following the maximum is almost identical in both cases.

In a glioblastoma (GS9L) rat tumor model, the contrast medium concentration in the tumor was compared at a low flow rate for two administration times. To this end, 5 □| of cell suspension (10⁶ glioblastoma 9L cells) was inoculated stereotactially into the brain of male Fischer rats. On day 11 after the inoculation, a dimeric contrast medium (Isovist 300, Bayer Schering Pharma, Berlin) was administered intravenously to the animals in a dose of 2 g of iodine/kg of body weight, and a CT examination was performed. The tumor-bearing animals were divided into two groups (n=3) by lot. The contrast medium was intravenously administered to animals of group 1 within 3 minutes and to those of group 2 within 6 minutes. Flow rates of about 0.55 or 0.28 ml/minute were produced therefrom. CT images of the rat head were made at the points in time 0, 1, 2, . . . 10, 12, 15 and 20 minutes after the beginning of the injection. The DynEva equipment software was used for evaluation. An ROI in vital tumor tissue and the skin was shown for each point in time, and the mean HU value was converted into the corresponding iodine concentration.

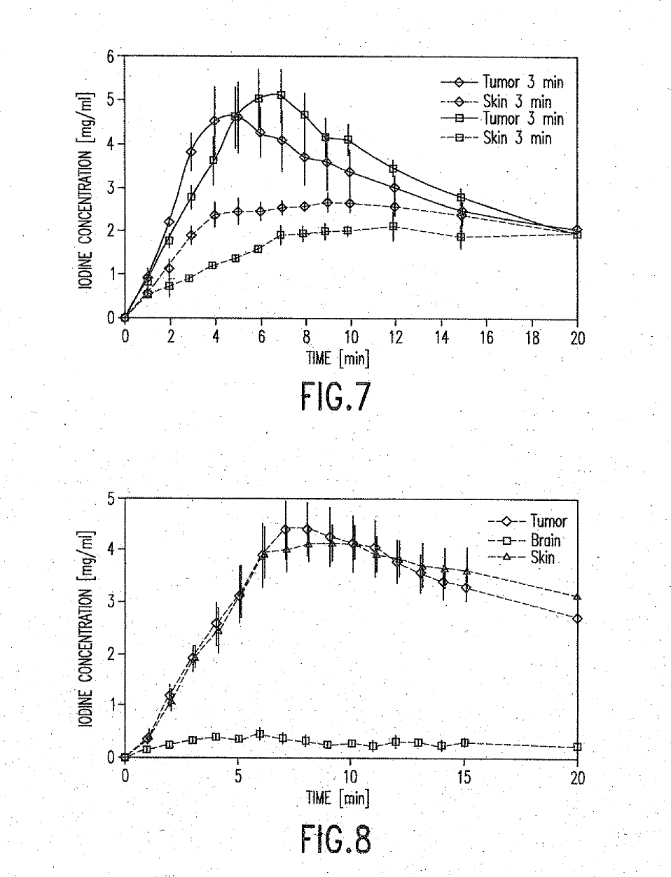

In both groups, a clear concentration maximum is visible in the time plot approximately one minute after the end of the administration. In the period between 4 and 5 minutes (3 minutes of infusion) or between 6 and 7 minutes (6 minutes of infusion), a short plateau phase is reached, in which the contrast medium concentration is changed only by a minimum amount (FIG. 7). The iodine concentration in the skin increases up to about 1 minute after the end of the contrast medium administration to values around 2 mgl/ml and remains at this level. The tumor-to-skin concentration ratio therefore reaches a maximum in the area of the plateau phase of the iodine concentration in the tumor.

Another possibility for modification of the contrast medium concentration is the use of two-phase or multi-phase injection protocols, in which the flow rate is changed during the administration. In this case, a portion is administered as a bolus with a high flow rate, followed by an infusion with decreasing or low flow. The target of this diagram is to keep the contrast medium concentration as constant as possible over an extended period. Simulations and an experimental trial on CT angiography in pigs showed that the vascular contrasting with bi- and multi-phase injections can be modified (18). In the ideal case, the iodine concentration in the vessels does not have any short peak but rather a plateau of up to 70 seconds. However, the maximum iodine concentration is also associated with a drop by about 20% (18). This diagram can basically also be transmitted to the tumor concentration. With respect to the contrast medium-enhanced radiation therapy, this makes possible specifically an extension of the time window for the introduction of the radiation dose, but it is also associated with a significant reduction of the local iodine concentration and thus the local radiation dose.

The contrast medium can also be intraarterially introduced into the vessel as in interventional angiography. To this end, a catheter is positioned in front of the outlet of the vascular section of interest. In the GS9L rat tumor model, the contrast medium concentration in the tumor was studied at a low flow rate for an intraarterial administration in 4 animals. To this end, the carotid artery was catheterized on day 10 after inoculation of the tumor cells, and a cannula was placed in the internal carotid artery (19). Via the latter, Isovist 300 was administered in a dosage of 2 g of iodine within 6 minutes. CT images of the rat head were made at the points in time 0, 1, 2, . . . 15 and 20 minutes after the beginning of the injection. For evaluation, the DynEva equipment software was used. The mean absorption in the tumor, adjacent healthy brain areas and in adjacent skin areas was determined for each point in time and converted into the corresponding iodine concentration. The iodine concentration in the tumor shows a maximum with a plateau phase about 1 minute after the end of the contrast medium administration (FIG. 8). Relative to an intravenous administration, the plateau phase is extended to 60-120 seconds, and the drop in iodine concentration is slower. During the contrast medium administration phase, iodine accumulates analogously in the tumor and reaches a plateau after the end of the latter. In the range between 6 and 10 minutes, the iodine concentration in the skin is less than in the tumor. In the healthy brain tissue, only very low iodine concentrations<0.5 mg/ml were observed.

Trials showed that the dynamic tumor contrast medium concentration can be modified by the flow rate in an intravenous administration. A pronounced concentration maximum in the tumor was observed, however, in both animal models, for both contrast medium classes (monomeric and dimeric compounds) and independently of the type of administration. At low flow rates, the maximum concentration in the tumor increases; the time span between the end of the contrast medium administration, and the peak is also increased. At very low flow rates, a plateau phase is formed in the animal model from about 60 s with high, almost constant contrast medium concentrations. By two- or multi-phase administration schemes, this plateau phase can be extended. The contrast medium concentration in the tumor thus drops significantly, however. Plateau phases up to 120 s can be achieved with an intraarterial injection.

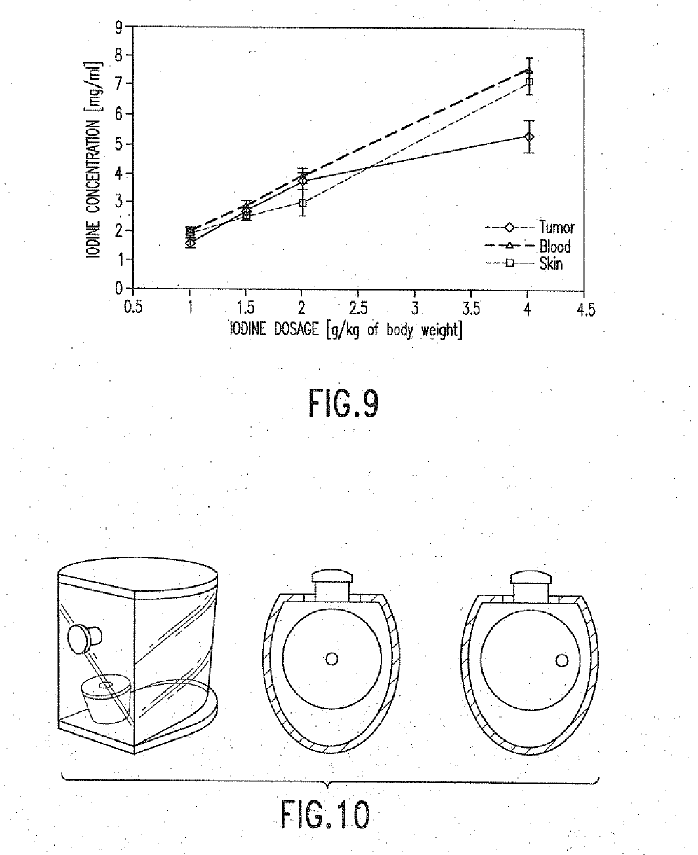

The second parameter for increasing the contrast medium concentration in the tumor is the contrast medium dosage. As a standard dose for the CT tumor diagnosis, 300 mg of iodine applies per kg of body weight. In the dosage range that is advantageous for tumor therapy (>1 gI/kg of body weight), no clinical data are present. To examine the connections between dosage and iodine concentration in tumors, an animal-trial study was therefore performed on a glioblastoma rat tumor model (see above). After a positive MRT tumor diagnosis, the animals were divided into 3 groups by lot. As a contrast medium, Isovist 300 was used, which was administered intravenously over 6 minutes. Group 1 (n=9) contained 1 mg of iodine/kg of body weight, group 2 (n=5) contained 2 mg of iodine/kg of body weight, and 4 mg of iodine/kg of body weight was administered to group 3 (n=9). After the beginning of the injection, CT images of the rat head were produced. The evaluation of the CT data was carried out in the contrast medium tumor concentration maximum (8 minutes after the beginning of the injection) with the CT equipment software. For each animal, an ROI in the tumor, the carotid artery, and the scalp was shown, and the mean HU values were converted into the corresponding iodine concentration. The result shows an increase in the iodine concentration with the dosage in all areas. While the increase in the blood vessels takes place almost linearly with the contrast medium dose, a flattening of the concentration increase for dosages of more than 2 g of iodine/kg of body weight is shown in the tumor. At the same time, the iodine concentration greatly increases in the skin that is well supplied with blood (FIG. 9). In the tumor area, a saturation, which is a function of tumor vascularization and the proportion of necrotic areas, is accordingly achieved at a dosage of about 2 g of iodine/kg of body weight. For higher dosages, the iodine concentration increases disproportionately in the vessels and the skin. In the contrast medium-enhanced radiation therapy, this results in a corresponding increase in the skin or vascular dose. A use of contrast medium dosages of greater than 2 g of iodine/kg of body weight results—in addition to a moderate increase in the tumor dose—in a disproportionate, intolerable increase of the absorbed radiation dose in healthy tissue.

The animal-trial studies show that the radiation dose should be incorporated within as short a time window as possible up to a maximum of 60 s (i.v.) or 120 s (i.a.) for the contrast medium-enhanced radiation therapy. Comparable data in humans are not present to date. The contrast medium flow was matched to human ratios in the animal-trial studies, so that the connections can be transferred between contrast medium flow and contrast medium dynamics. Based on the recommendations of the manufacturer (</=1.5 g of iodine/kg of body weight) and the concentration characteristic in the tumor and healthy tissue, the contrast medium dose is limited above. In the animal model, an optimum dosage of 2 g of iodine/kg of body weight was observed.

### X-Ray Tubes

In radiation therapy, the kV therapy with x-ray tubes up to 300 kV acceleration voltage is now no longer used. X-ray tubes are used in the area of diagnosis in CT devices, angiography units, C-arm, mammography units and conventional table-x-ray devices. With the exception of mammography, all x-ray tubes have a wolfram anode and are operated to a maximum of 140 kV acceleration voltage. This energy range is very well suited for the contrast medium-enhanced radiation therapy.

The maximum hardware requirements are made on tubes for CT devices. The erratic technological developments in the clinical CT were characterized by a reduction of the rotation times of 1.0 to 0.27 s and the use of always wider detectors with up to 320 parallel lines Both parameters required a very high photon flow rate φ and thus a very high power of the tubes. For these devices, tubes with a focal-spot power of 70-120 kW are necessary. The essential power-limiting factor is the storage and discharge of the focal-spot heat that is produced in the generation of the x-ray radiation. High-power tubes can therefore be produced in principle only with rotating anodes, in which the focal-spot heat that is produced is distributed. The storage capacity of the anode is a function of the material properties, focal-spot size and power, as well as of the radius and the rotational speed of the anode itself (20). The energy that is stored in the anode is indicated in Mega Heat Units (MHU). The energy must be drawn off by an effective cooling mechanism; the cooling power is indicated in MHU/minute. The combination of storage capacity and cooling power determines the maximum power-time product (critical load curve), which can be produced without the occurrence of cooling times. With respect to the kV therapy, the critical load curve determines the maximum dose power that can be achieved within a time window. A volatile increase of the cooling power was achieved by the introduction of the rotating envelope tube technology, as it is used in the Siemens Straton tubes (20). The heat that is produced is not dissipated here by radiation but rather by convection. Additional examples of high-power tubes are the MRC tubes of Philips (21) and the Megacool tubes of Toshiba (22).

TABLE 2
Heat Storage Capacity, Cooling Rate and Power
of Current High-Power Tubes (20, 21, 23).

| | Siemens Straton | Philips MRC | Toshiba Megacool |
|---|---|---|---|
| Storage Capacity | 0.6 | 8.0 | 7.5 |
| [MHU] | | | |
| Cooling Rate | >5.0 | 1.6 | 1.4 |
| [MHU/minute] | | | |
| Power [kW] | 80 | 120 | 72 |

### High-Power X-Ray Tubes for Contrast Medium-Enhanced Radiation Therapy

In current radiotherapy, the total dose is administered in fractionated form with only a few special exceptions (radiosurgery). There are various irradiation schemes (standard fractionation, hyperfractionation, and hypofractionation). In by far the largest number, a standard fractionation with single doses of between 1.8 and 3 Gy is used (24). The dose rate that is used is approximately 3 Gy/minute, i.e., a single dose is administered within 1 to 2 minutes. The total-brain irradiation for treating multiple brain metastases forms a special case. In contrast to conventional treatment schemes, the radiation in this case is not focused on the minor area but rather irradiates the total brain. The fractionation is carried out with single doses of between 2 and 3 Gy (24).

The requirements of the radiation source for clinical contrast medium-enhanced radiation therapy are specified by the contrast medium dynamics. A basic requirement is as high a dose rate as possible, which makes possible the introduction of a single dose within the shortest time, to ensure an optimum adaptation between tumor-contrast medium concentration and irradiation. As a function of the contrast medium administration parameters, a time window of up to at most 120 s is available for this purpose. In the latter, a start can be made from a maximum, time-stable, contrast medium tumor concentration. In this time range, a single dose of 1.8-3 Gy has to be administered.

The Most modern CT devices have ideal requirements for the contrast medium-enhanced radiation therapy based on their high-power tubes as well as the rotation principle. A device-specific dosimetric parameter is the Computed Tomography Dose index (CTDI) in the rotation center of the CT gantry. In addition to the dose in the layer, the latter also takes into consideration the dose contributions of the branches over a range of 100 mm:

$${CTDI}_{100 , air} = \frac{1}{M \cdot S} {\int }_{- 50 mm}^{50 mm} D ( z ) d z , \qquad ( Equation 5 )$$

whereby M represents the number of lines and S represents the layer thickness. The CTDI_{100,air} is also referred to as an air-kerma dose and describes the nominal dose of the CT devices in the rotation center. In now common devices, the latter lies between 7 mGy/100 mAs (80 kV) and 30 Gy/100 mAs (140 kV). A specified time Window of 30, 60 and 90 s and a corresponding tube power yield air-kerma doses of between 0.42 and 2.7 Gy/100 mAs (Table 3).

TABLE 3
Air-Kerma Dose (CTDI_{100, Vol}) at 80 and 140
kV and Radiation Times of 30, 60 and 100 s.

| | Time (s) | | | | | |
|---|---|---|---|---|---|---|
| | 30 s | | 60 s | | 100 s | |
| Voltage (kV) | 80 | 140 | 80 | 140 | 80 | 140 |
| Air-Kerma | 0.21 | 0.9 | 0.42 | 1.8 | 0.7 | 3.0 |
| (Gy/100 mAs) | | | | | | |

The air-kerma dose disregards the weakening of the radiation by the object itself that occurs in practice. Dose data in phantoms are therefore to be preferred. As a standard, the Computed Tomography Dose Index (CTDI_{100,Vol})| applies. The latter is measured in standardized, tissue-like phantoms (16 cm head/32 cm body) and is used for information on dose reference values. In this case, the dose in the center of the phantom (D_{Center}) is weighted with the dose in the periphery (D_{Periphery)}:

$${CTDI}_{100 , Vol} = \frac{1}{M \cdot S \cdot P} \cdot ( \frac{1}{3} {\int }_{- 50 mm}^{50 mm} {D}_{Center} ( z ) d z + \frac{2}{3} {\int }_{- 50 mm}^{50 mm} {D}_{Periphery} ( z ) d z ) \qquad ( Equation 6 )$$

The CTDI_{100,Vol} is standardized to the total collimation M*S and the pitch P and is indicated for each study on the CT device. Table 4 contains the CTDI_{100,Vol}|data of the CT device Sensation 64 (Siemens Medical, Erlangen) for a collimation of 28.8 mm and an irradiation without a table feed. The differences of the region being considered (head/body) illustrate the influence of the phantom size on the dose. For applications in the area of the radiation therapy, the CTDI is only conditionally reliable.

TABLE 4
CTDI_{100, Vol} at 80 and 140 kV, 60 and 100 s of Radiation
Time and 0 mm of Table Feed for the Siemens Sensation 64.

| | Time (s) | | | | | |
|---|---|---|---|---|---|---|
| | 30 s | | 60 s | | 100 s | |
| Voltage (kV) | 80 | 140 | 80 | 140 | 80 | 140 |
| CTDI_{100, Vol}\| Body | 0.05 | 0.32 | 0.11 | 0.63 | 0.18 | 1.05 |
| (Gy/100 mAs) | | | | | | |
| CTDI_{100, Vol}\| Head | 0.24 | 0.63 | 0.24 | 1.26 | 0.40 | 2.09 |
| (Gy/100 mAs) | | | | | | |

The above-mentioned limitations of the CTDI-dose sizes required an experimental determination of the CT radiation dose. To this end, measurements were taken on a Siemens Sensation 64, which is operated with a high-power tube (Straton Z). For simulation of realistic conditions, an anthropomorphic head phantom, which copies the absorption properties of the head, was used (QRM GmbH, Möhrendorf). Since not the kerma dose but rather the water energy dose represents the basis of clinical dosimetry in radiation therapy, a correspondingly calibrated ionization chamber was used as a detector (Type 31010, PTW, Freiburg, calibration certificate No. 0609797). With the latter, the local dose was determined in the radiation field (chamber volume=0.125 cm³). The chamber was positioned either centrally or peripherally over a special insert in the phantom (FIG. 10). As a reference, a measurement in air was used

The CT equipment software makes possible a continuous irradiation of a maximum of 100 s The dose measurements were taken for the time ranges 30, 60 and 100 s, in which the maximum applicable water energy dose was determined. To this end, the maximum mAs product that can be selected in the CT device for this time range was selected. Via the total collimation (28.8 mm) and the table feed (0 mm), the irradiation field was matched to a realistic clinical target column. The gantry turn-around time was 1 s. Table 5 shows the results of the dose measurements. With increasing radiation time, the maximum possible mAs product and thus the nominal dose rate in air drops from 5.5 Gy/minute to 3.2 Gy/minute (140 kV). At the same time, the local total dose in the target volume increases from 1.2 to 2.4 Gy (140 kV). For therapy, suitable radiation doses>1.8 Gy can be achieved with a tube voltage of 140 kV. Within 60 s, an administration of 2 Gy is also possible in a target volume that is central in the head. Clinically, the occurrence of peripheral lesions and thus target volumes is more probable. In the phantom measurements, up to 2.6 Gy (100 s of radiation time) could be incorporated in peripheral anatomical positions.

In the total brain irradiation, the volume to be irradiated comprises the total brain with the inclusion of the lamina cribrosa, the base of the skull with the basal cisterns as well as the cervical vertebrae 1 and 2. The volume that is to be covered is thus considerably larger than in the conventional radiation therapy. The geometric width of the CT beam in the direction of the body axis is limited by collimators on the detector Width. CT devices of the newest generation have very large volume detectors, with detector widths of 40 mm (Phillips Brilliance 64) up to 160 mm (Toshiba Aquilion One). In the case of very wide detectors, the total target volume is found within the fan beam or cone beam. The total brain irradiation can thus be produced without table feed. When using less wide detectors or radiation geometries, the target volume can be covered by a sequential or spiral-shaped irradiation with table feed. An extension of the radiation time is associated, however, so that an introduction of a single dose of 2 Gy with commercially available tubes still cannot be carried out within the desired time window of 120 s. An alternative to avoiding the table feed could show hardware optimizations, thus the layer collimation for therapy could be matched to the size of the brain. Also, new techniques for adaptive detector covers such as 4DAS (Siemens Medical Solutions; Erlangen) could be used. The power requirements of x-ray tubes for the contrast medium-enhanced total brain irradiation are especially high based on the large target volume. Based on the contrast medium dynamics, a single dose of 1.8-3 Gy has to be administered within a shortest possible time window. This can be carried out only with high-power tubes.

In the clinical radiation therapy, the desired dose and its distribution is calculated in the irradiation planning, whereby the nominal values of the linear accelerator (dose rate, dose distribution) are present in the simulation software or are determined by reference dosimetry. In contrast to this, no comparable planning programs exist for the energy range up to 140 keV. The dose distribution for a standard CT can be simulated, however, with the Monte Carlo-based software ImpactMC (Vamp GmbH, Erlangen). The simulated dose distribution for the above-described measuring arrangement shows significant superelevation of the initial dose in comparison to the central range (FIG. 11). In, addition to the absorption properties of bones, this can also be attributed to the radiation geometry of the diagnostic CT device. By suitable measures, such as, e.g., filtering or x-y collimation, the initial dose can be considerably reduced without significantly influencing the dose in the center (7). The described requirements regarding dose rate thus also have their validity in a CT device that is optimized for therapy. Regarding the hardware portion, a patent with use of x-ray optical modules was filed with the Patent Office under AZ. 10 2007 018 102.9 on Apr. 16, 2007.

### Device for Combination of Contrast Medium Concentration and Therapy

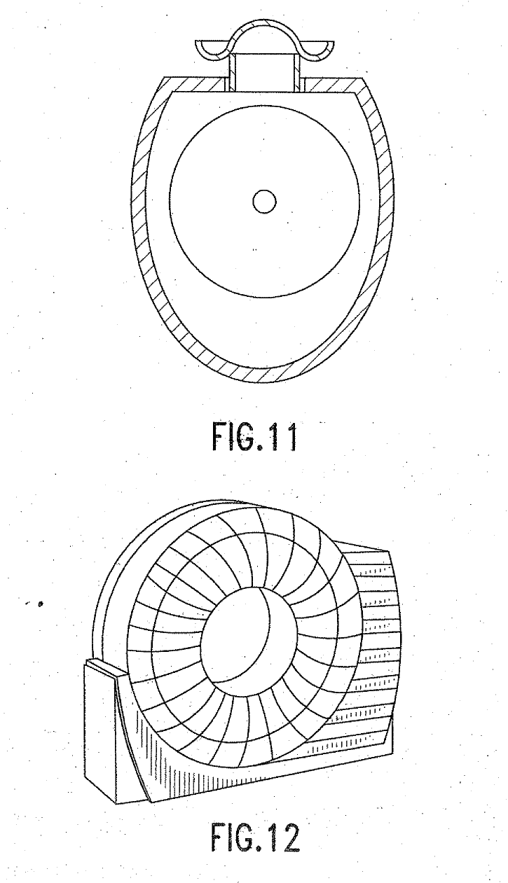

The implementation of a clinical contrast medium-enhanced radiation therapy is specified by the contrast medium kinetics in the tumor area. The latter varies as a function of the tumor anatomy and physiology. The therapy therefore requires an individual determination of the therapy time window. The study of the individual tumor contrast medium kinetics can be implemented within the scope of the irradiation planning before the actual beginning of therapy. It has to be assumed, however, that the specific kinetics during the therapy clearly change. Such changes are detected by an online monitoring of the contrast medium concentration in the target volume and surrounding tissues, and the therapies are matched accordingly. A combination of online monitoring and radiation therapy can be produced with a 2-tube system, such as the dual-source CT (FIG. 12). While one x-ray tube introduces the radiation dose into the target volume, the other is used for simultaneous imaging. In the images that are generated in this case, the contrast medium concentration can be shown site-resolved via the absorption values (HU values). Based on these data, the therapy can be modified according to the contrast medium kinetics. Also spatial changes of the target volume (tumor expansion, tumor positioning) are thus visible. The radiation therapy can be adapted for any therapy session to changes in the contrast medium kinetics and the target volume. Another important advantage of the contrast medium concentration of real-time monitoring is online therapy control. In a contrast medium concentration in the tumor or in healthy tissue that deviates from the preset values, the irradiation can be broken off immediately. Conversely, the irradiation can also be started only when a contrast medium concentration threshold value is reached.

The x-ray tubes are fastened to a rotating gantry. Relative to the imaging tubes, a detector is necessary for receiving the absorption data. To minimize the influence of the scattered radiation of the therapy tubes on the imaging, the distances of the radiator can be optimized. With a 2-tube system, the tubes are offset by 90°.

A device for contrast medium-enhanced radiation therapy, has to have at least 2 x-ray tubes, whereby a tube for determining the contrast medium kinetics and/or target volume positioning (tracking) is used in real time. For therapy, at least one high-power x-ray tube is used. The latter has to correspond to the above-described requirements of high-power tubes for radiation therapy. A minimum requirement is the introduction of a radiation dose of 2 Gy into the target volume within 60 s. This requires an air-kerma dose rate of at least 4.5 Gy/minute. This can be carried out with x-ray tubes with a power of at least 80 kW and/or a thermal anode-continuous power (10 minutes) of at least 7 kW. The determination of the contrast medium absorption or concentration and the target volume tracking can be carried out with an x-ray tube with low power (>40 kW).

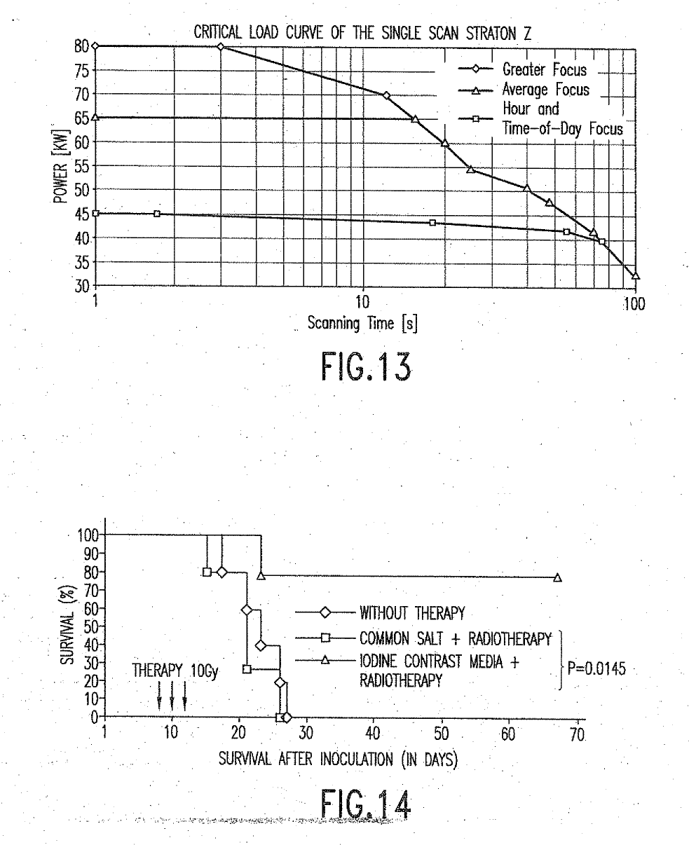

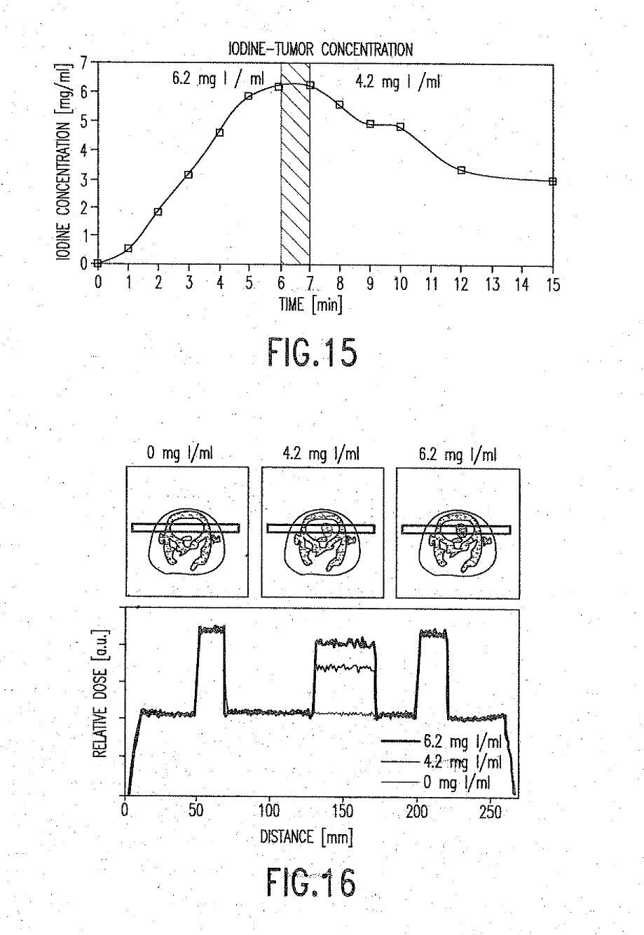

A device for contrast medium-enhanced radiation therapy can be based on the commercially available Dual-Source CT Siemens Definition (Siemens Medical Solutions, Erlangen, Germany). This device has two Straton Z high-power tubes, which can be operated simultaneously. With respect to the contrast medium-enhanced radiation therapy, a tube can be used for the introduction of the radiation dose, while the second tube is used for imaging. The Straton Z-tube has a power of 80 kW. The thermal anode-continuous power is 4.9 kW, or 7 kW within 10 minutes. The critical load curve shows a maximum power of about 47.5 kW at a scanning time of 60 seconds, and 33 kW at 100 seconds (FIG. 13). With these power parameters, a contrast medium-enhanced radiation therapy can be carried out.

### EXAMPLES

### 1. Contrast Medium-Enhanced Radiation Therapy on the Animal Model

The therapeutic effectiveness of the contrast medium-enhanced radiation therapy was examined on a glioblastoma (GS9L) rat tumor model. To this end, male Fisher rats were inoculated stereotactically with 5 □I of cell suspension (10⁶ glioblastoma 9L cells) in the brain. After 8 days, the tumor growth was ensured by means of MRT, and the animals were divided into 3 groups (n=5). The animals of group 1 received no therapy and were used as a control. Animals of groups 2 and 3 passed through a CT radiation therapy with a total dose of 18 Gy at a tube voltage of 140 kV (Volume Zoom, Siemens Medical Solutions, Erlangen). Six therapy sessions were performed (2 per day at an interval of 4 hours), and one dose of 3 Gy was administered to the animals in each case. The radiation was limited to the tumor using the layer collimators and introduced within 90 s, which corresponds to a tumor-dose rate of 2 Gy/minute. Before each therapy session, a dimeric contrast medium (Isovist 300, Bayer Schering Pharma, Berlin) in a dose of 2 g of iodine/kg of body weight was administered to the animals of group 2. The latter was administered intravenously, within 6 minutes. With this application scheme, a maximum iodine-tumor concentration in a short plateau phase of between 6 and 7 minutes is achieved (FIG. 14). In this time range, beginning with the end of the contrast medium injection, the introduction of the radiation dose took place. Before the irradiation, an isotonic common salt solution was infused in the animals of group 3 instead of contrast medium.

The animals whose tumor was not treated or was irradiated only in the presence of common salt died within 14 days after the therapy. The animals that were irradiated in the presence of contrast media had a considerable therapeutic advantage, which can be directly detected in the survival of the animals (FIG. 14). The test was completed after 10 weeks.

### 2. Dose Simulations on the Rat Model

The combination of contrast medium dynamics and dose rate was examined based on dose simulations. As a basis, the time plot of the contrast medium concentration in the tumor on the GS9L animal model was used in an administration protocol optimized for the contrast medium-enhanced radiation therapy (2 g of iodine/kg of body weight; intravenous administration within 6 minutes). FIG. 13 shows the time plot of the iodine concentration in tumors of an animal. In the plateau phase directly after the end of the contrast medium administration (6-7 minutes), the mean iodine concentration is 6.2 mg/ml. When taking a period of 9 minutes (6-15 minutes) into consideration, 4.2 mg of iodine/ml in the tumor is found on average.

For dose simulation, the Monte Carlo-based Software Impact MC was used. As a starting point, CT images of the rat head were used directly before (0 mg/ml) and after contrast medium administration. In Impact MC, a concentration of 4.2 or 6.2 mgl/ml was set for the contrast media. For simulation, the standard device data of the Siemens Volume Zoom were used at 140 kV. In the resulting dosage charts, the increase of the tumor dose can be detected with the iodine concentration (FIG. 16). Based on the dose profile, the latter can be quantified. In a radiation therapy without contrast media, no increase in the radiation dose in the tumor in comparison to the adjacent tissue can be detected when using an unmodified diagnostic CT device. The tumor dose, however, increases with the iodine concentration by the factor 1.5 (4.2 mgl/ml) or 1.8 (6.2 mg). The radiation dose that is inserted into the skull is independent of the contrast medium concentration.

These results demonstrate the influence of contrast medium dynamics and dose rates on the tumor dose. An efficient contrast medium-enhanced radiation therapy must therefore take place with as high a dose rate as possible. The latter can be carried out only with high-power x-ray tubes.

3. Comparison of the Contrast Medium-Enhanced Radiation Therapy with Gold Standard

For examination of the therapeutic effectiveness of the contrast medium-enhanced radiation therapy with a high dose rate, the latter was compared to the therapeutic standard, i.e., a radiation therapy on the linear accelerator. To this end, a glioblastoma (GS9L) rat tumor model was used. Male Fischer rats were stereotactially inoculated with 5 □l of cell suspension (10⁶ glioblastoma 9L cells) in the brain. After 8 days, the to growth was ensured by means of MRT, and the animals were divided into 3 groups. The animals of group 1 (n=6) obtained no therapy and were used as a control. Animals of group 2 (n=5) were treated on the linear accelerator (Novalis, Brain Lab AG, Feldkirchen) at 2 MV with a total dose of 18 Gy. This dose was administered to 3 in fractions of 6 Gy each on days 9, 10 and 11 after inoculation. The animals of group 3 passed through a contrast medium-enhanced radiation therapy on days 9-11 with a total dose of 18 Gy and a tube voltage of 140 kV (Volume Zoom, Siemens Medical Solutions, Erlangen). Six therapy sessions were carried out (2 per day at 4-hour intervals) and in each case a dose of 3 Gy was administered to the animals within 90 seconds. This corresponds to a tumor-dose rate of 2 Gy/minute. Before each therapy session, a dimeric contrast medium (Isovist 300, Bayer Schering Pharma, Berlin) was administered intravenously within 6 minutes in a dose of 2 g of iodine/kg of body weight. In both therapy schemes, the radiation was limited to the total brain using the collimators.

The animals whose tumors were treated on the linear accelerator showed only a slight survival advantage relative to the control group. In contrast to this, 2 of 6 animals that passed through a contrast medium-enhanced therapy showed a significant therapy effect. The latter can be detected directly in the survival time of the animals (FIG. 17). In the animal model, the contrast medium-enhanced radiation therapy with a high dose rate shows a therapeutic advantage relative to the standard therapy on the linear accelerator. It can be assumed from this that the latter becomes even clearer when high-power CT devices are optimized for radiation therapy.

In the process of the invention, Table 5 shows usable contrast media.

TABLE 5

| Trade Name | Active Ingredient | Manufacturer |
|---|---|---|
| Ultravist | Iopromide | BSP |
| Solutrast | Iopamidol | Bracco |
| Iopamiron | Iopamidol | BSP |
| Omnipaque | Iohexol | BSP |
| Accupaque | Iohexol | GE Healthcare |
| Omnipaque Injection | Iohexol | GE Healthcare |
| Isovist | Iotrolan | BSP |
| Optiray | Ioversol | Covidien |
| Imagopaque | Iopentol | GE Healthcare |
| Visipaque | Iodixanol | GE Healthcare |
| Iomeron | Iomeprol | Bracco |
| Xenetix | Iobitridol | Guerbet |
| Oxilan | Ioxilan | Guerbet |
| Hexabrix | Ioxaglinic Acid | Guerbet |
| MultiHance | Gadobenate Dimeglumine | Bracco |
| Gadovist, Gadograf | Gadobutrol | BSP |
| Omniscan | Gadodiamide | GE Healthcare (Daiichi in Japan) |
| Magnevist, Magnograf | Gadopentetate Dimeglumine | BSP |
| Dotarem, Magnescope (JP) | Gadoterate Meglumine | Guerbet (Termuo in JP) |
| ProHance | Gadoteridol | Bracco (Eisei in JP) |
| OptiMARK | Gadoversetamide | Covidien |
| Primovist, Eovist | Gadoxetic Acid | BSP |
| Vasovist | Gadofosveset | BSP |
| Resovist | Ferucarbotran (USAN) | BSP, Meito JP |
| Endorem/Feridex | Dextran-Coated Ferumoxide | BSP, Eiken (J), Guerbet (EU) |
| Teslascan | Mangafodipir Trisodium | GE Healthcare |

The invention comprises in particular:

1. A device for radiation-therapeutic treatment that consists of an x-ray CT unit or an x-ray-angiography unit or an orthovolt-x-ray unit with, in each case, at least one x-ray radiation source, characterized in that the x-ray source consists of a high-power x-ray tube, which makes the radiation doses that are necessary for the therapy sessions applicable at one time.

2. A device for radiation-therapeutic treatment of tissues that are provided with a photoelectrically activatable contrast medium by means of an x-ray CT unit or by means of an x-ray-angiography unit or orthovolt-x-ray unit with, in each case, at least one x-ray radiation source, wherein the x-ray source consists of a high-power x-ray tube, which makes the radiation doses that are necessary for the therapy sessions applicable at one time.

3. A device according to Item 1 or 2, wherein the system can be operated both in the diagnostic mode and in the therapy mode.

4. A device according to Item 3, wherein in the diagnostic mode, the system can apply the beam as a fan beam or cone beam, and in the therapy mode, the beam can be configured in a compressible manner so that the target object is preferably illuminated.

5. A device according to Items 1-4, wherein the x-ray unit that is used has at least two x-ray tubes, whereby by means of at least one high-power x-ray tube, the radiation dose that is necessary for therapy can be administered in a time window that is established based on the contrast medium concentration by the time-synchronous measurement by means of another x-ray tube that is operated in the diagnostic mode.

6. A process for determining the optimum time window in the contrast medium-enhanced radiation therapy, wherein a device according to Items 1-5 is used, and the optimum therapy time window is selected in preliminary tests in which the time window of the irradiation is set such that a higher contrast medium concentration than in the irradiated healthy tissue is present in the target area.

7. A process for determining the optimum time window in the contrast medium-enhanced radiation therapy, wherein a device according to Items 1-5 is used, and the optimum therapy time window is selected at the same time as the therapy in which the device contains at least two tubes, and one tube operates in the diagnostic mode and the second operates in the therapy mode, and the time window of the therapeutic irradiation is set such that a higher contrast medium concentration than in the irradiated healthy tissue is present in the target area.

8. A process according to Items 6 and 7, wherein the therapy window is between 1 s and 300 s.

9. A process according to Items 6 and 7, wherein the therapy window is less than or equal to 200 s.

10. A process according to Items 6 and 7, wherein the therapy window is less than 100 s.

11. A process according to Items 6-10, wherein before the therapy, a photoelectrically activatable contrast medium is administered, and wherein the dose rate is adjusted to the pharmacokinetics in the target volume and in the irradiated healthy tissue.

12. A process according to Items 6-11, wherein before and during the therapy, a photoelectrically activatable contrast medium selected from the group that consists of iopromide, iopamidol, iopamidol, iohexol, iohexol, iohexol, iotrolan, ioversol, iopentol, iodixanol, iomeprol, iobitridol, ioxilan, ioxaglinic acid, gadobenate dimeglumine, gadobutrol, gadodiamide, gadopentetate dimeglumine, gadoterate meglumine, gadoteridol, gadoversetamide, gadoxetic acid, gadofosvset, ferucarbotran (USAN), dextran-coated ferumoxide and mangafodipir trisodium is administered.

13. A process according to Item 12, wherein the dose of the contrast medium is greater than 0.1 g of I/kg, but less than 4 g of I/kg.

14. A process according to Items 6 and 7, wherein the x-ray dose rate is greater than 1 Gy/minute.

15. A process according to items 6 and 7, wherein the x-ray dose rate is greater than 2 Gy/minute.

16. A process for contrast medium-enhanced radiation therapy of tumors, in which a photoelectrically activatable contrast medium is administered to the patient, wherein

a device according to Items 1-5 is used,

and the optimum therapy time window is selected in preliminary tests in which the time window of the irradiation is set such that a higher contrast medium concentration than in the irradiated healthy tissue is present in the target area,

and wherein it is irradiated for the corresponding length of time.

17. A process for contrast medium-enhanced radiation therapy of tumors, in which a photoelectrically activatable contrast medium is administered to the patient, wherein

a device according to Items 1-5 is used,

and the optimum therapy time window is selected at the same time as the therapy, in which the device contains at least two tubes, and one tube operates in the diagnostic mode, and the second operates in the therapy mode, and the time window of the therapeutic irradiation is set such that a higher contrast medium concentration than in the irradiated healthy tissue is present in the target area,

and wherein it is irradiated for the corresponding length of time.

18. A process according to Item 16 or 17, wherein a contrast medium is used that is selected from the group that consists of iopromide, iopamidol, iopamidol, iohexol, iohexol, iohexol, iotrolan, ioversol, iopentol, iodixanol, iomeprol, iobitridol, ioxilan, ioxaglinic acid, gadobenate dimeglumine, gadobutrol, gadodiamide, gadopentetate dimeglumine, gadoterate meglumine, gadoteridol, gadoversetamide, gadoxetic acid, gadofosveset, ferucarbotran (USAN), dextran-coated ferumoxide and mangafodipir trisodium.

19. A process according to Items 16, 17 or 18, wherein the therapy window is between 1 s and 120 s.

20. A process according to Items 16, 17 or 18, wherein the therapy window is less than or equal to 100 s.

21. A process according to items 16, 17 or 18, wherein the therapy window is less than 300 s.

22. A process according to Item 16, wherein a photoelectrically activatable contrast medium is administered before the therapy, and wherein the dose rate is adjusted to the pharmacokinetics in the target volume and in the irradiated healthy tissue.

The contrast media that are mentioned in Table 5 are examples of contrast media that are suitable for the process.

23. A process according to Item 16 or 17, wherein the dose of the contrast medium is greater than 0.1 g of I/kg, but less than 4 g of I/kg.

24. A process according to Item 16 or17, wherein the dose of the contrast medium is greater than or equal to 0.1 mmol of Gd/kg but less than 5 mmol of Gd/kg.

25. A process according to Item 16 or 17, wherein the x-ray dose rate is greater than 1 Gy/minute.

26. A process according to Item 16 or 17, wherein the x-ray dose rate is greater than 2 Gy/minute.

### REFERENCES

1. Verellen, D., Ridder, M. D., Linthout, N., Tournel, K., Soete, G., Storme, G. Innovations in Image-Guided Radiotherapy. Nat Rev Cancer 2007; 7 (12): 949-960.
2. Speck, U. Kontrastmittel in der Radiologie—Röntgen and MRT [Contrast Media in Radiology—X-Ray and MRT]. Radiologie up to date [Up-to-Date Radiology] 2003 (1): 81-94.
3. Callisen, H. H., Norman, A., Adams, F. H. Absorbed Dose in the Presence of Contrast Agents during Pediatric Cardiac Catheterization. Med Phys 1979; 6(6): 504-509.
4. Fairchild, R. G., Bond, V. P. Photon Activation Therapy. Strahlentherapie [Radiation Therapy] 1984; 160(12): 758-763.
5. Santos Mello, R., Callisen, H., Winter, J., Kagan, A. R., Norman, A. Radiation Dose Enhancement in Tumors with Iodine. Med Phys 1983; 10(1), pp. 75-78.
6. Iwamoto, K. S., Cochran, S. T., Winter, J., Holburt, E., Higaschida, R. T., Norman, A. Radiation Dose Enhancement Therapy with Iodine in Rabbit VX-2 Brain Tumors. Radiother Oncol 1987; 8(2): 161-170.
7. Iwamoto, K. S., Norman, A., Kagan, A. R., Wollin, M., Olch, A., Bellotti, Ingram, M., Skillen, R. G. The CT Scanner as a Therapy Machine. Radiother Oncol 1990; 19(4): 337-343.
8. Rose, J. H., Norman, A., Ingram, M., Aoki, C., Solberg, T., Mesa, A. First Radiotherapy of Human Metastatic Brain Tumors Delivered by a Computerized Tomography Scanner (CTRx). Int J Radiat Oncol Biol Phys 1999; 45(5): 1127-1132.
9. Adam, T. F., Elleaume, H., Joubert, A., Biston, M. C., Charvet, A. M., Baloso, J., Le Bas, J. F., Esteve, F. Synchrotron Radiation Therapy of Malignant Brain Glioma Loaded with an Iodinated Contrast Agent: First Trial on Rats Bearing F98 Gliomas. Int J Radiat Oncol Biol Phys 2003; 57(5): 1413-1426.
10. Sterzing, F., Herfarth, K., Debus, J. IGRT with Helical Tomotherapy—Effort and Benefit in Clinical Routine. Strahlenther Onkol 2007; 183 Spec No. 2: 35-37.
11. Mesa, A. V., Norman, A., Solbert, T. D., Demarco, J. J., Smathers, J. B. Dose Distributions Using Kilovoltage X-Rays and Dose Enhancement from Iodine Contrast Agents. Phys Med Biol 1999; 44(8): 1955-1968.
12. Boudou, C., Balosso, J., Esteve, F., Elleaume, H. Monte Carlo Dosimetry for Synchrotron Stereotactic Radiotherapy of Brain Tumors. Phys Med Biol 2005; 50(20); 4841-4851.
13. Rutten, A., Prokop, M. Contrast Agents in X-Ray Computed Tomography and its Applications in Oncology. Anticancer Agents Med Chem 2007; 7(3): 307-316.
14. Adam, J. F., Bison, M. C., Joubert, A., Charvet, A. M., Le Bas, J. F., Esteve, F., Elleaume, H. Enhanced Delivery of Iodine for Synchrotron Stereotactic Radiotherapy by Means of Intracarotid Injection and Blood-Brain Barrier Disruption: Quantitative Iodine Biodistribution Studies and Associated Dosimetry. Int J Radiat Oncol Biol Phys 2005; 61(4): 1173-1182.
15. NIST. (http://physics.nist.gov/PhysRefData/XrayMassCoef/cover.html).
16. Fong, P. M., Keil, D. C., Does, M. D., Gore, J. C. Polymer Gels for Magnetic Resonance Imaging of Radiation Dose Distributions at Normal Room Atmosphere. Phys Med Biol 2001; 46(12): 3105-3113.
17. Dunne, A. A., Mandic, R., Ramaswamy, A., Plehn, S., Schulz, S., Lippert, B. M., Moll, R., Werner, J. A. Lymphogenic Metastatic Spread of Auricular VX2 Carcinoma in New Zealand White Rabbits. Anticancer Res 2002; 22(6A): 3273-3279.
18. Bae, K. T., Tran, H. Q., Heiken, J. P. Multiphasic Injection Method for Uniform Prolonged Vascular Enhancement at CT Angiography: Pharmacokinetic Analysis and Experimental Porcine Model. Radiology 2000; 216(3): 872-880.
19. Bullard, D. E., Saris, S. C., Bigner, D. D. Carotid Artery Injections in 40 g to 99 g Fischer Rats: Technical Note and Evaluation of Blood Flow by Various Injection Techniques. Neurosurgery 1984; 14(4): 406-411.
20. Schardt, P., Deuringer, J., Freudenberger, J., Hell, E., Knupfer, W., Mattern, D., Schild, M. New X-Ray Tube Performance in Computed Tomography by Introducing the Rotating Envelope Tube Technology. Med Phys 2004; 31(9): 209-2706.
21. Schmidt, T., Behling, R. MRC: A Successful Platform for Future X-Ray Tube Development. Medica Mundi 2000; 44(2): 50-55.
22. Toyomasa, H. Development of a Large-Capacity, High-Cooling-Rate X-Ray Tube for Ultra-High Speed CT Systems. Medikaru, Rebyu 1999; 23(4): 77-79.
23. Toshiba Medical Systems. http://www.toshiba-europe.com/medical/medicalp.asp?PageID=1131&PRODUCT ID=1621.2008.
24. DGN. Leitlinien [Guidelines] der Deutschen Gesellschaft für Neurologie. Deutsche Gesellschaft für Neurologie; 2008.

1. Device for radiation-therapeutic treatment that consists of an x-ray CT unit or an x-ray-angiography unit or an orthovolt-x-ray unit with in each case at least one x-ray radiation source, characterized in that the x-ray source consists of a high-power x-ray tube, which makes the radiation doses that are necessary for the therapy sessions applicable at one time.

2. Device for radiation-therapeutic treatment of tissues that are provided with a photoelectrically activatable contrast medium by means of an x-ray CT unit or by means of an x-ray-angiography unit or orthovolt-x-ray unit with, in each case, at least one x-ray radiation source wherein the x-ray source consists of a high-power x-ray tube, which makes the radiation doses that are necessary for the therapy sessions applicable at one time.

3. Device according to claim 1 or 2, wherein the system can be operated both in the diagnostic mode and in the therapy mode.

4. Device according to claim 3, wherein in the diagnostic mode, the system can apply the beam as a fan beam or cone beam, and in the therapy mode, the beam can be configured in a compressible manner such that the target object is preferably illuminated.

5. Device according to claims 1-4, wherein the x-ray unit that is used has at least two x-ray tubes, whereby by means of at least one high-power x-ray tube, the radiation dose that is necessary for therapy can be administered in a time window that is established based on the contrast medium concentration by the time-synchronous measurement by means of another x-ray tube that is operated in the diagnostic mode.

6. Process for determining the optimum time window in the contrast medium-enhanced radiation therapy, wherein a device according to claims 1-5 is used, and the optimum therapy time window is selected in preliminary tests in which the time window of the irradiation is set such that a higher contrast medium concentration than in the irradiated healthy tissue is present in the target area.

7. Process for determining the optimum time window in the contrast medium-enhanced radiation therapy, wherein a device according to claims 1-5 is used, and the optimum therapy time window is selected at the same time as the therapy in which the device contains at least two tubes, and one tube operates in the diagnostic mode and the second operates in the therapy mode, and the time window of the therapeutic irradiation is set such that a higher contrast medium concentration than in the irradiated healthy tissue is present in the target area.

8. Process according to claims 6 and 7, wherein the therapy window is between 1 s and 300 s.

9. Process according to claims 6-8, wherein before the therapy, a photoelectrically activatable contrast medium is administered, and wherein the dose rate is adjusted to the pharmacokinetics in the target volume and in the irradiated healthy tissue.

10. Process according to claims 6-9, wherein before and during the therapy, a photoelectrically activatable contrast Medium selected from the group that consists of iopromide, iopamidol, iopamidol, iohexol, iohexol, iohexol, iotrolan, ioversol, iopentol, iodixanol, iomeprol, iobitridol, ioxilan, ioxaglinic acid, gadobenate dimeglumine, gadobutrol, gadodiamide, gadopentetate dimeglumine, gadoterate meglumine, gadoteridol, gadoversetamide, gadoxetic acid, gadofosveset, ferucarbotran (USAN), dextran-coated ferumoxide and mangafodipir trisodium is administered.

11. Process according to claim 10, wherein the dose of the contrast medium is greater than 0.1 g of I/kg, but less than 4 g of I/kg.

12. Process according to claims 6 and 7, wherein the x-ray dose rate is greater than 1 Gy/minute.

13. Process for contrast medium-enhanced radiation therapy of tumors, in which a photoelectrically activatable contrast medium is administered to the patient, wherein
a device according to claims 1-5 is used,
and the optimum therapy time window is selected in preliminary tests in which the time window of the irradiation is set such that a higher contrast medium concentration than in the irradiated healthy tissue is present in the target area,
and wherein it is irradiated for the corresponding length of time.

14. Process for contrast medium-enhanced radiation therapy of tumors, in which a photoelectrically activatable contrast medium is administered to the patient, wherein
a device according to claims 1-5 is used,
and the optimum therapy time window is selected at the same time as the therapy in which the device contains at least two tubes and one tube operates in the diagnostic mode, and the second operates in the therapy mode, and the time window of the therapeutic irradiation is set such that a higher contrast medium concentration than in the irradiated healthy tissue is present in the target area
and wherein it is irradiated for the corresponding length of time.

15. Process according to claim 13 or 14, wherein the therapy window is between 1 s and 120 s.

16. Process according to claim 13, wherein a photoelectrically activatable contrast medium is administered before the therapy, and wherein the dose rate is adjusted to the pharmacokinetics in the target volume and in the irradiated healthy tissue.
